(19) United States
(12) Patent Application Publication
(10) Pub. No.: US 2025/0069201 A1
(43) Pub. Date: Feb. 27, 2025

(54) GUIDEWIRE ARTIFACT SUPPRESSION METHOD AND DEVICE, IVUS SYSTEM, AND STORAGE MEDIUM

(71) Applicant: MICROIMAGING (SHENZHEN) MEDICAL EQUIPMENT CO., LTD, Shenzhen, Guangdong (CN)

(72) Inventors: Qing HE, Shenzhen (CN); Zhihua HE, Shenzhen (CN)

(21) Appl. No.: 18/725,001

(22) PCT Filed: Nov. 9, 2022
(86) PCT No.: PCT/CN2022/130793

§ 371 Date: Jun. 27, 2024

(30) Foreign Application Priority Data
Dec. 31, 2021 (CN) 202111672204.6

(51) Int. Cl. G06T 5/70; G06T 5/20; G06T 5/60

(52) U.S. Cl. CPC G06T 5/70; G06T 5/20; G06T 5/60; G06T 2207/10132; G06T 2207/20084; G06T 2207/30021; G06T 2207/30101

(57) ABSTRACT

The present application relates to a guidewire artifact suppression method and device, an IVUS system, and a storage medium. The guidewire artifact suppression method comprises the following steps: acquiring first scan line data; when guidewire artifact information is present in the first scan line data, performing filtering processing and tissue information extraction processing on the first scan line data to obtain second scan line data; and performing logarithmic processing on the second scan line data on the basis of a dynamic coefficient, so as to obtain third scan line data for reconstructing an ultrasonic image, wherein the dynamic coefficient is obtained on the basis of the second scan line data, a position of the guidewire artifact information and reference scan line data, and the reference scan line data is obtained according to first scan line data that has no guidewire artifact information therein.

### CROSS REFERENCE TO RELATED DISCLOSURE

The present application claims priority to Chinese Patent Disclosure with No. 202111672204.6, entitled “Guidewire Artifact Suppression Method and Device, IVUS System, and Storage Medium”, and filed on Dec. 31, 2021, the content of which is expressly incorporated herein by reference in its entirety.

### TECHNICAL FIELD

The present disclosure relates to the field of ultrasonic imaging technology, and particularly to a guidewire artifact suppression method and apparatus, an IVUS system, and a storage medium.

### BACKGROUND

Intravascular ultrasound imaging, also referred to as Intravascular Ultrasound, i.e., IVUS technology, refers to mounting a miniature ultrasound probe at the front end of a catheter. The catheter is inserted deep into the blood vessel by means of professional technologies to explore the tissue structure of the blood vessel, which is a relatively effective, direct, and high-quality ultrasound diagnostic technology at the present stage. For the mechanical rotation type IVUS system, a rotating motor drives the single-element transducer in the conductor to rotate. During the rotation process, the single-element transducer periodically transmits ultrasonic excitation signals and receives ultrasonic echo signals.

### SUMMARY

In one aspect of the present disclosure, a guidewire artifact suppression method is provided, including:
acquiring first scan line data;
when the first scan line data includes guidewire artifact information, performing filtering processing and tissue information extraction processing on the first scan line data to obtain second scan line data; and
performing logarithmetics processing on the second scan line data based on a dynamic coefficient to obtain third scan line data for reconstructing an ultrasound image, wherein the dynamic coefficient is obtained based on the second scan line data, a position of the guidewire artifact information and reference scan line data, and the reference scan line data is obtained based on first scan line data excluding the guidewire artifact information.

In an embodiment, the method may further include:
performing the filtering processing and the tissue information extraction processing on the first scan line data when the first scan line data excludes the guidewire artifact information, to obtain fourth scan line data; and
performing the logarithmetics processing on the fourth scan line data based on a fixed coefficient to obtain fifth scan line data for reconstructing the ultrasound image.

In an embodiment, the performing the filtering processing on the first scan line data may include:
performing bandpass filtering processing on the first scan line data when the first scan line data excludes the guidewire artifact information; and
performing the bandpass filtering processing and bandstop filtering processing on the first scan line data respectively when the first scan line data includes the guidewire artifact information.

In an embodiment, the method may further include:
processing a time domain, a frequency domain or a time-frequency domain of each piece of first scan line data to obtain a characteristic value of each piece of first scan line data;
processing each characteristic value by a classifier to obtain a classification result; and
determining whether each piece of first scan line data includes the guidewire artifact information according to the classification result.

In an embodiment, the first scan line data includes a plurality of pieces of sub-data; and the processing each characteristic value by the classifier includes:
processing characteristic values of first N pieces of sub-data by the classifier, wherein a value of N is obtained according to setting parameters and a catheter structure of a ultrasound system.

In an embodiment, the first scan line data includes a plurality of pieces of sub-data; and the processing each characteristic value by the classifier includes:
processing characteristic values of the plurality of pieces of sub-data in sequence by the classifier until a guidewire artifact determination event occurs, wherein the guidewire artifact determination event includes that the first scan line data is determined to include the guidewire artifact information based on a classification result corresponding to a piece of sub-data.

In an embodiment, the method may further include:
determining the reference scan line data as a standard template;
determining theoretical data of a scanning point according to the position of the guidewire artifact information, the standard template, and actual data of the scanning point in the first scanning line data including the guidewire artifact information; and
determining the dynamic coefficient based on the theoretical data and the actual data.

In an embodiment, the method may further include:
acquiring a fitting function by a neural network algorithm model;
processing, by the fitting function, the position of the guidewire artifact information, the actual data of the scanning point in the first scanning line data including the guidewire artifact information, and the reference scanning line data having a scanning angle adjacent to a scanning angle of the first scanning line data including the guidewire artifact information to obtain the dynamic coefficient.

In an embodiment, the performing the logarithmetics processing on the second scan line data based on the dynamic coefficient to obtain the third scan line data for reconstructing the ultrasound image is based on a following formula:

$$y = log ( k * x + 1 ) ,$$

where y denotes the third scan line data, k denotes the dynamic coefficient, and x denotes the second scan line data.

In one aspect of the present disclosure, a guidewire artifact suppression apparatus is provided, including:
a data cache module configured to acquire first scan line data;
a processing module configured to, when the first scan line data includes guidewire artifact information, perform filtering processing and tissue information extraction processing on the first scan line data to obtain second scan line data;
a logarithmetics module configured to perform logarithmetics processing on the second scan line data based on a dynamic coefficient to obtain third scan line data for reconstructing an ultrasound image, wherein the dynamic coefficient is obtained based on the second scan line data, a position of the guidewire artifact information and reference scan line data, and the reference scan line data is obtained based on the first scan line data excluding the guidewire artifact information.

In one aspect of the present disclosure, an intravascular ultrasound (IVUS) system is provided, including a processor and a memory storing a computer program, the processor, when executing the computer program, implements the method of any one of the above embodiments.

In another aspect of the present disclosure, a computer-readable storage medium is provided, on which a computer program is stored, the computer program, when executed by a processor, causes the processor to implement the method of any one of the above embodiments.

### BRIEF DESCRIPTION OF THE DRAWINGS

Other limitations, purposes and advantages of the present disclosure will become more obvious by reading the detailed description of non-limiting embodiments with reference to the following accompanying drawings.

FIG. 1 is a flow chart I showing a guidewire artifact suppression method according to an embodiment.

FIG. 2 is a schematic diagram of a guidewire artifact according to an embodiment.

FIG. 3 is a flow chart showing steps of determining whether there exists guidewire artifact information in each piece of first scan line data according to an embodiment.

FIG. 4 is a flow chart II showing a guidewire artifact suppression method according to an embodiment.

FIG. 5 is a before-and-after comparison diagram of an ultrasound image containing a guidewire artifact processed by the method of the present disclosure according to an embodiment.

FIG. 6 is a flow chart I showing steps of determining a dynamic coefficient according to an embodiment.

FIG. 7 is a flow chart II showing steps of determining a dynamic coefficient according to an embodiment.

FIG. 8 is a structural block diagram of a guidewire artifact suppression apparatus according to an embodiment.

FIG. 9 is an internal structure diagram of an IVUS system according to embodiment.

### DETAILED DESCRIPTION

In order to make the above purpose, technical solution and advantages of the present disclosure more obvious and easier to understand, the present disclosure will be further elaborated below with reference to the accompanying drawings and embodiments. It should be appreciated that the specific embodiments described herein are merely used for explaining the present disclosure, rather than limiting the present disclosure.

During the implementation process, the inventors discovered that there are at least the following problem in the conventional technology: IVUS images presented by the conventional mechanical rotation type IVUS system have guidewire artifacts.

In view of this, for the above technical problem, it is necessary to provide a guidewire artifact suppression method and apparatus, an IVUS system, and a storage medium that can suppress the guidewire artifacts in the IVUS images.

In an embodiment, as shown in FIG. 1, a guidewire artifact suppression method is provided, which is applied to an IVUS system as an example for description, and the method may include the following steps.

S110: first scan line data is acquired.

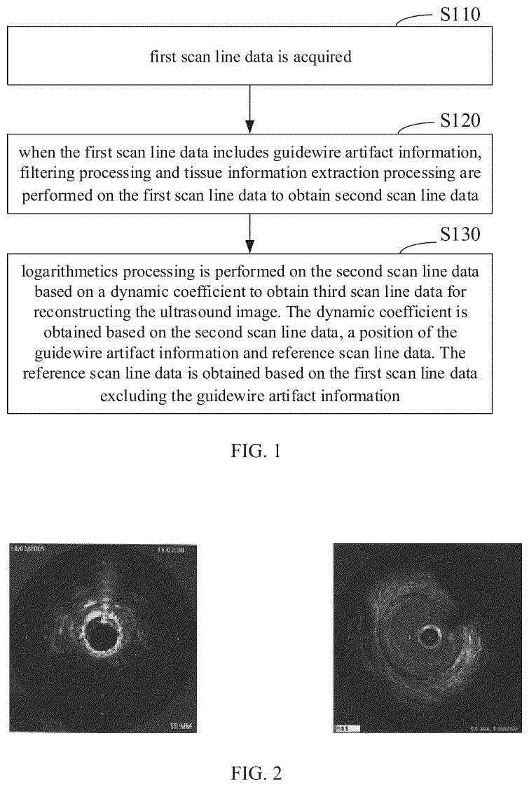

The first scan line data refers to a digital signal, which is obtained by converting a reflected ultrasonic echo signal received by an ultrasonic probe into an electrical signal, and converting the electrical signal through an analog-to-digital conversion. The first scan line data may be radio frequency signal data or the like. The ultrasound probe is a component of an ultrasound imaging system. Optionally, the ultrasound imaging system is an intravascular ultrasound imaging system of a mechanical rotation type. The ultrasound probe of the ultrasound system has a mechanically rotating structure, and the guidewire may be located on one side of the catheter when the probe rotates and scans. Accordingly, the guidewire may be scanned by the ultrasound probe, resulting in guidewire artifacts on the IVUS image. As shown in FIG. 2, the guidewire may appear as a bright echo signal on the intravascular ultrasound image, a strong echo point shadow of the guidewire and an acoustic shadow behind the guidewire can be seen in a lumen. Reference can be made to graphic examples at the 12 o'clock direction in the left picture and the 2 o'clock direction in the right picture in FIG. 2.

The first scan line data at each scanning angle may be acquired by any means in the art. In an example, the first scan line data transmitted from the ultrasound probe may be directly received. In another example, the ultrasound probe stores the first scan line data collected at each scanning angle in a data buffer, and when the guidewire artifact suppression processing is required, the data can be directly extracted from the data buffer. The ultrasonic probe can collect echo signals including vascular tissue information at different scanning angles, and the echo signals are converted into the first scanning line data at different scanning angles through the digital-to-analog conversion. In other words, the first scan line data transmitted by the ultrasound probe may be acquired in real time, or a plurality of pieces of first scan line data or all first scan line data transmitted by the ultrasound probe may be acquired.

In the embodiment, the real-time acquisition of the first scan line data transmitted by the ultrasound probe is taken as an example for description. In this cases, the acquired first scan line data is current first scan line data.

S120: when the first scan line data includes guidewire artifact information, filtering processing and tissue information extraction processing are performed on the first scan line data to obtain second scan line data.

Any means in the art may be utilized to perform the filtering processing and tissue information extraction processing on the first scan line data. In an example, when the first scan line data excludes the guidewire artifact information, bandpass filtering processing is performed on the first scan line data. For the first scan line data including the guidewire artifact information, the bandpass filtering processing and bandstop filtering processing are respectively performed on the first scan line data in sequence, in order to reduce the signal energy generated by the guidewire artifact. The bandpass filtering processing may be FIR bandpass filtering processing, and the bandstop filtering processing may be FIR bandstop filtering processing. It should be noted that when the first scan line data excludes the guidewire artifact information, the bandpass filtering processing is performed on the first scan line data, which can improve a signal-to-noise ratio of the radio frequency signal data. For the first scan line data including the guidewire artifact information, the bandstop filtering processing is added on the basis of the above-mentioned bandpass filtering processing, which can further suppress the signal energy near the IVUS excitation signal frequency based on the bandstop filtering processing, thereby reducing the signal energy generated by the guidewire artifact.

It should be noted that other filtering processing modes may also be used as long as the signal energy generated by the guidewire artifact can be reduced and the signal-to-noise ratio of the first scan line data including the guidewire artifact information can be improved.

The vascular tissue information extraction can be performed by using any technical means in the art, and can be performed based on a time domain, a frequency domain, or a time-frequency domain. For example, the technical means may be an orthogonal demodulation algorithm in an ultrasound image reconstruction based on the time domain, or a wavelet extraction method based on the time-frequency domain.

In the embodiment, for the current first scan line data obtained in step S110, when the current first scan line data includes the guidewire artifact information, the FIR bandpass filtering processing and FIR band group filtering processing are performed on the current scan line data, and then the vascular tissue information is extracted by means of the orthogonal demodulation algorithm to obtain the current second scan line data.

S130: logarithmetics processing is performed on the second scan line data based on a dynamic coefficient to obtain third scan line data for reconstructing the ultrasound image. The dynamic coefficient is obtained based on the second scan line data, a position of the guidewire artifact information and reference scan line data. The reference scan line data is obtained based on the first scan line data excluding the guidewire artifact information.

For the second scan line data, since most of the ultrasonic signals are blocked by the guidewire, the vascular tissue signal behind the guidewire may be greatly attenuated. In addition, due to the different relative positions of the guidewire, the vascular tissue and the probe, the attenuation degree of the vascular tissue signal may be different. Accordingly, the logarithmetics dynamic coefficient at the moment may need to be adjusted in real time during the second scan line data processing.

It should be noted that when the logarithrnetics processing is performed on each piece of second scan line data, i.e., the current second scan line data, the dynamic coefficient thereof is obtained based on the position of the guidewire artifact information, actual data of the current second scan line data and the reference scan line data. The reference scan line data refers to scan line data obtained by performing the logarithmetics processing on the first scan line data adjacent to the current first scan line data and excluding the guidewire artifact information. In other words, if the current first scan line data is the k-th piece of first scan line data, scan line data obtained by performing the logarithmetics processing on the previous most recent first scan line data excluding the guidewire artifact information is the reference scan line data. That is, if the (k-1)-th piece of first scan line data is the first scan line data excluding the guidewire artifact information, the scan line data obtained after the logarithmetics processing is the reference scan line data. If the (k-1)-th piece of first scan line data includes the guidewire artifact information, the (k-2)-th piece of first scan line data needs to be determined, and so on, to determine the most recent first scan line data excluding the guidewire artifact information, Since the vascular tissue has continuity on the IVUS image, the second scan line data may be processed based on reference scan line data having a scanning angle adjacent to the scanning angle of the second scan line data to obtain third scan line data. Through the above-mentioned logarithmetics processing, various vascular tissues can be clearly displayed within a smaller grayscale variation range, which helps users to find lesions more quickly.

In a specific example, in the step of performing the logarithmetics processing on the second scan line data based on the dynamic coefficient to obtain the third scan line data for reconstructing the ultrasound image, the third scan line data is obtained based on the following formula:

$$y = log ( k * x + 1 ) ,$$

where y denotes the third scan line data, k denotes the dynamic coefficient, and x denotes the second scan line data.

It should be appreciated that the above-mentioned guidewire artifact suppression method can be applied to process the current first scan line data, that is, when the ultrasound probe transmits any piece of first scan line data (current first scan line data), the piece of first scan line data is processed by the above-mentioned guidewire artifact suppression method. When the ultrasound probe transmits the next piece of first scan line data, the next piece of first scan line data is processed by the above-mentioned guidewire artifact suppression method, and so on, until all the first scan line data are processed.

In some embodiments, the guidewire artifact suppression method may be adopted to process a plurality of pieces of first scan line data simultaneously. That is, when a plurality of sets of first scan line data are acquired, a plurality of processing resources may be allocated to process the first scan line data simultaneously.

With the above-mentioned guidewire artifact suppression method, the guidewire artifact signal with higher energy can be effectively suppressed by performing corresponding filtering processing on each piece of first scan line data when the first scan line data includes the guidewire artifact information. At the same time, the logarithmetic processing is performed on the second scan line data based on the dynamic coefficient, considering the continuity of the vascular tissue image, which enhances the vascular tissue signal in the guidewire artifact region and further suppresses the guidewire artifact information. Meanwhile, the authenticity and validity of the data are guaranteed to a large extent, which contributes to improving the display effect of lesions in the guidewire artifact region. In other words, the logarithrnetics processing is performed on the second scan line data based on the appropriate adjustment of the dynamic coefficient, which can compress and expand the intensity variation difference of different tissue signals, so that as many different vascular tissues as possible can be clearly displayed to the user within a smaller grayscale variation range.

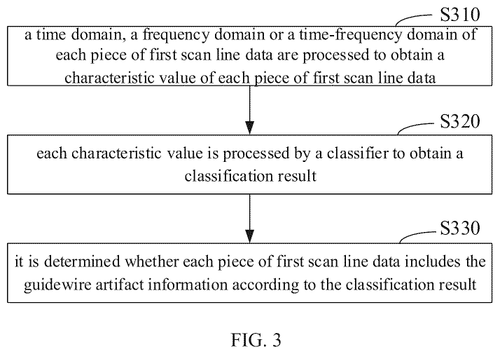

In an embodiment, as shown in FIG. 3, the step of determining whether each piece of first scan line data includes the guidewire artifact information may include following steps.

S310: a time domain, a frequency domain or a time-frequency domain of each piece of first scan line data is processed to obtain a characteristic value of each piece of first scan line data.

The processing mode for the frequency domain or time-frequency domain may be the processing mode commonly used in the art, such as FIR filtering processing, wavelet decomposition processing, etc.

S320: each characteristic value is processed by a classifier to obtain a classification result.

The first scan line data is also a radio frequency (RF) signal, the characteristic value is extracted from the RF signal in real time, and the characteristic value extracted in real time is input into the classifier for identification. Different classifiers can be selected according to different development platform resources. In a specific example, the classifier may be a Bayesian classifier, a neural network classifier, or a deep belief network classifier, etc.

S330: it is determined whether each piece of first scan line data includes the guidewire artifact information according to the classification result,

It is possible to directly determine whether the guidewire artifact information exists in the first scan line data through the classification result.

In an embodiment, the first scan line data includes a plurality of pieces of sub-data, and the step of processing the characteristic value by the classifier may include:
characteristic values of the first N pieces of sub-data are processed by the classifier. The value of N is obtained according to setting parameters and a catheter structure of the ultrasound system. N is a natural number greater than 1. It should be appreciated that the first scan line data including a plurality of pieces of sub-data refers to a plurality of pieces of first scan line data acquired at different moments.

Due to the catheter structure of the mechanical rotary IVUS, the position of the guidewire is relatively close to the position of the ultrasound probe, so for the scan line data affected by the guidewire artifact, the characteristic of the guidewire artifact will be reflected in the first N pieces of sub-data. Accordingly, when the characteristic value is extracted, it is not necessary to extract all the data in the entire scan line data, while it is only necessary to extract and identify the first N pieces of data in the first scan line data. The above method can greatly improve the real-time performance of the algorithm and reduce the algorithm delay. The value of N can be obtained according to the setting parameters and the catheter structure of the ultrasound system. It should be noted that the first scan line data may be radio frequency signal data.

In an example, in order to further improve the efficiency, the characteristic values can be extracted synchronously during the scan line data collection process, and the extracted characteristic values are inputted into the classifier in real time for identification. Once the guidewire artifact is identified, search is stopped immediately and an identification result is outputted. Accordingly, there is no need to wait until the characteristic values of the first N pieces of data are fully extracted before outputting the result.

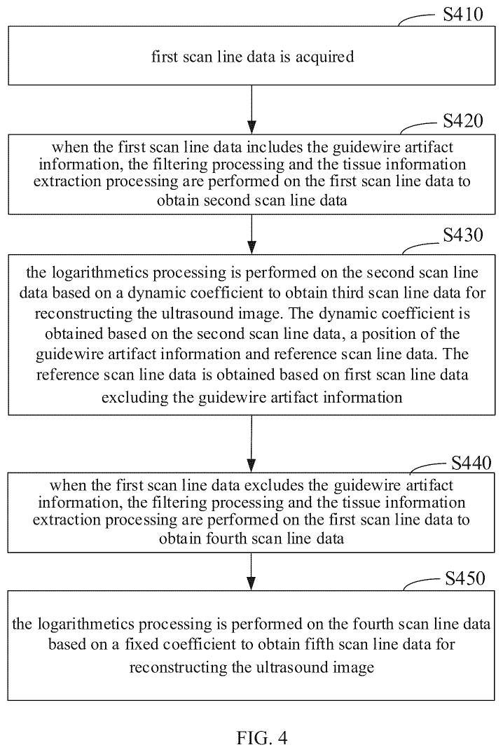

In an embodiment, as shown in FIG. 4, a guidewire artifact suppression method is provided, which may include the following steps.

S410: first scan line data is acquired.

S420: when the first scan line data includes the guidewire artifact information, the filtering processing and the tissue information extraction processing are performed on the first scan line data to obtain second scan line data.

S430: the logarithmetics processing is performed on the second scan line data based on a dynamic coefficient to obtain third scan line data for reconstructing the ultrasound image. The dynamic coefficient is obtained based on the second scan line data, a position of the guidewire artifact information and reference scan line data. The reference scan line data is obtained based on first scan line data excluding the guidewire artifact information.

The method may further include the following steps.

S440: when the first scan line data excludes the guidewire artifact information, the filtering processing and the tissue information extraction processing are performed on the first scan line data to obtain fourth scan line data.

Any means in the art may be adopted to perform the filtering processing and the tissue information extraction processing on the first scan line data. In a specific example, when the first scan line data excludes the guidewire artifact information, the FIR bandpass filtering processing is performed on the first scan line data. Through the filtering processing, the FIR bandpass filtering processing is performed on the first scan line data excluding the guidewire artifact information, and accordingly, the signal-to-noise ratio of the RF data is improved.

S450: the logarithmetics processing is performed on the fourth scan line data based on a fixed coefficient to obtain fifth scan line data for reconstructing the ultrasound image.

For the fourth scan line data, a fixed coefficient is adopted to perform the logarithmetics processing on the fourth scan line data. The fixed coefficient may be determined before the IVUS system starts scanning according to the system itself and requirements of the user for the brightness of the ultrasound image. As for the step of performing the logarithmetics processing on the scan line data excluding the guidewire artifact information, reference can be made to the process of performing the logarithmetics processing on the scan line data including the guidewire artifact information. Furthermore, the fixed coefficient is determined according to the image brightness requirement of the ultrasound image.

In another specific example, the step of performing the logarithmetics processing on the fourth scan line data based on the fixed coefficient to obtain the fifth scan line data for reconstructing the ultrasound image may be based on the following formula:

$$A = log ( a * B + 1 ) ;$$

where A denotes the fifth scan line data, B denotes the fourth scan line data, and a denotes the fixed coefficient.

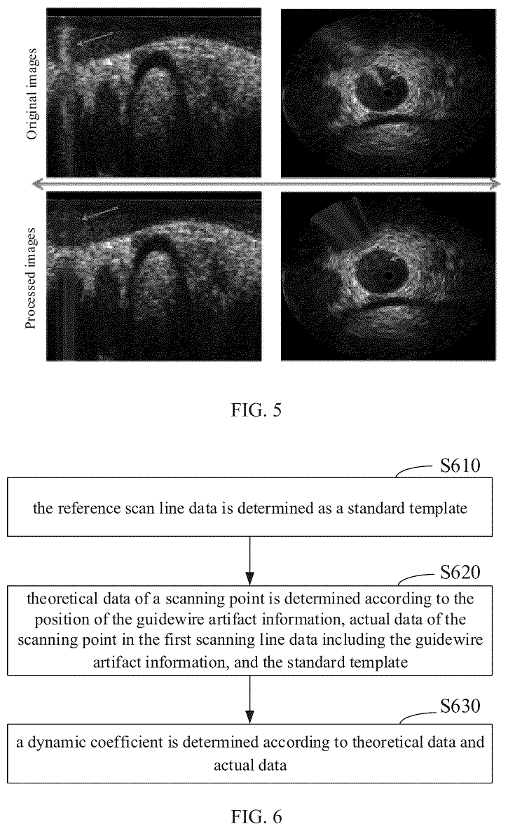

It should be noted that the specific step of the above-mentioned logarithmetics processing may include that: the logarithmetics processing is performed on each piece of sub-data in the second scan line data, and when each piece of sub-data in the second scan line data is traversed, the third scan line data is obtained. As for the specific processing step of the fourth scan line data, reference can be made to the step for the second scan line data. FIG. 5 shows original images without being processed by the guidewire artifact suppression method of the present disclosure and processed images obtained after the processing. As can be seen from FIG. 5, after the guidewire artifact is processed by the method of the present disclosure, the guidewire artifact can be significantly suppressed and the structure in the region can be clear.

In an embodiment, as shown in FIG. 6, the step of determining the dynamic coefficient may include following steps.

S610: the reference scan line data is determined as a standard template.

S620: theoretical data of a scanning point is determined according to the position of the guidewire artifact information, actual data of the scanning point in the first scanning line data including the guidewire artifact information, and the standard template.

S630: a dynamic coefficient is determined according to theoretical data and actual data.

In an embodiment, the step of determining the dynamic coefficient according to theoretical data and actual data may be based on the following formula:

$$k = f ( x , i , {line}_{near} ) ;$$

where k denotes the dynamic coefficient, x denotes the second scan line data, i denotes the position of the current RF data in the current scan line, and line, denotes a previous piece of scan line data that is not affected by the guidewire artifact.

Specifically, due to the continuity of the vascular tissue image, the current scan line data and the previous piece of scan line data not affected by the guidewire artifact may have a similar signal amplitude change trend. Therefore, the reference scan line data having a scanning angle adjacent to a scanning angle of the first scan line data including the guidewire artifact information can serve as a standard template to determine the function ƒ, and then the dynamic coefficient k is calculated in combination with the position of the guidewire artifact information and the current second scan line data. In other embodiments, the neural network technology may be introduced to perform linear fitting, and a more appropriate function ƒ may be obtained through large sample size training. For details, reference can be made to the relevant description of steps S710 and S720 below.

In an example, the reference scan line data is the previous piece of scan line data that is not affected by the guidewire artifact. In another example, the reference scan line data is the previous piece of scan line data that is not affected by the guidewire artifact and has a scanning angle that is adjacent to (the same as or similar to) the scanning angle of the first scan line data.

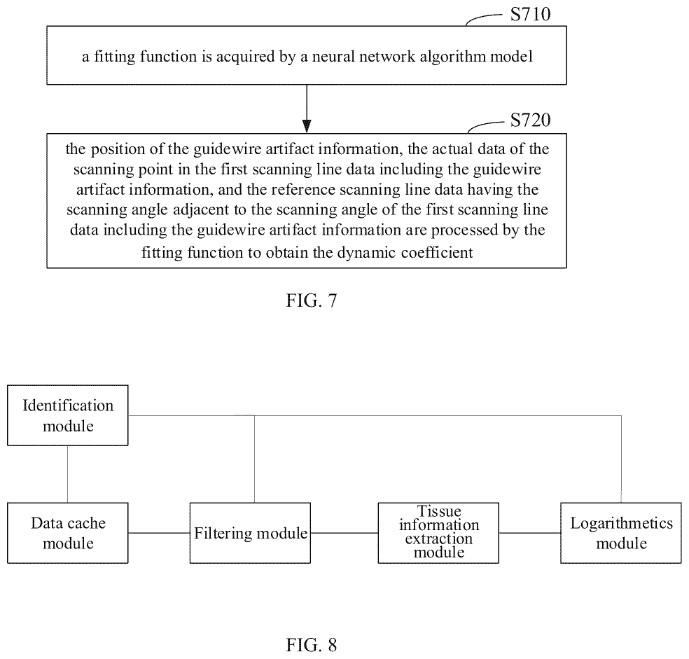

In an embodiment, as shown in FIG. 7, the step of determining the dynamic coefficient may include the following steps.

S710: a fitting function is acquired by a neural network algorithm model.

In an example, a neural network algorithm model is trained to obtain a fitting function, the output parameters of which are dynamic coefficients, and the input parameters of which may include the reference scan line data, the data value and position information of any data point in the processed scan line data.

S720: the position of the guidewire artifact information, the actual data of the scanning point in the first scanning line data including the guidewire artifact information, and the reference scanning line data having the scanning angle adjacent to the scanning angle of the first scanning line data including the guidewire artifact information are processed by the fitting function to obtain the dynamic coefficient.

In an example, the fitting function can be obtained by training any neural network algorithm model in the art. It should be noted that the fitting function is based on the dynamic coefficient, the actual data of the scanning point in the first scanning line data including the guidewire artifact information, and standard scanning line data having a scanning angle adjacent to the scanning angle of the first scanning line data including the guidewire artifact information.

It should be appreciated that although the steps in the flow charts of FIGS. 1 to 7 are displayed sequentially as indicated by arrows, these steps are not definitely executed sequentially in the order indicated by the arrows. Unless otherwise specified herein, there is no strict order limitation for the execution of these steps, and these steps may be executed in other orders. Moreover, at least part of the steps in FIGS. 1 to 7 may include multiple sub-steps or multiple stages. These sub-steps or stages are not definitely executed at the same moment, but can be executed at different moments. These sub-steps or stages are not definitely executed sequentially, but may be executed in turns or alternately with other steps or at least part of the sub-steps or stages of other steps.

In an embodiment, as shown in FIG. 8, a guidewire artifact suppression apparatus is provided, which may include:
a data cache module configured to acquire first scan line data;
a processing module configured to, when the first scan line data includes guidewire artifact information, perform filtering processing and tissue information extraction processing on the first scan line data to obtain second scan line data;
a logarithmetics module configured to perform logarithmetics processing on the second scan line data based on a dynamic coefficient to obtain third scan line data for reconstructing an ultrasound image, in which the dynamic coefficient is obtained based on the second scan line data, a position of the guidewire artifact information and reference scan line data, and the reference scan line data is obtained based on the first scan line data excluding the guidewire artifact information.

In an embodiment, the processing module is further configured to perform the filtering processing and the tissue information extraction processing on the first scan line data when the first scan line data excludes the guidewire artifact information, to obtain fourth scan line data.

The logarithmetics module is further configured to perform the logarithmetics processing on the fourth scan line data based on a fixed coefficient to obtain fifth scan line data for reconstructing the ultrasound image.

In an embodiment, the processing module is further configured to perform bandpass filtering processing on the first scan line data when the first scan line data excludes the guidewire artifact information, and perform the bandpass filtering processing and bandstop filtering processing on the first scan line data respectively when the first scan line data includes the guidewire artifact information.

In an embodiment, the guidewire artifact suppression apparatus may further include:
an extraction module configured to process a time domain, a frequency domain or a time-frequency domain of each piece of first scan line data to obtain a characteristic value of each piece of first scan line data;
a classification module configured to process each characteristic value by a classifier to obtain a classification result;
a determination module configured to determine whether each piece of first scan line data includes the guidewire artifact information according to the classification result.

In an embodiment, the first scan line data may include a plurality of pieces of sub-data, and the classification module is further configured to process characteristic values of the first N pieces of sub-data by the classifier, in which a value of N is obtained according to setting parameters and a catheter structure of the ultrasound system.

In an embodiment, the first scan line data may include a plurality of pieces of sub-data, and the classification module is further configured to process the characteristic values of the plurality of pieces of sub-data in sequence by the classifier until a guidewire artifact determination event occurs, in which the guidewire artifact determination event may include that the first scan line data is determined to include the guidewire artifact information based on a classification result corresponding to any piece of sub-data.

In an embodiment, the logarithmetics module is further configured to: determine the reference scan line data as a standard template; determine theoretical data of a scanning point according to the position of the guidewire artifact information, the standard template, and actual data of the scanning point in the first scanning line data including the guidewire artifact information; and determine the dynamic coefficient based on the theoretical data and the actual data.

In an embodiment, the logarithmetics module is further configured to: acquire a fitting function by a neural network algorithm model; process, by the fitting function, the position of the guidewire artifact information, the actual data of the scanning point in the first scanning line data including the guidewire artifact information, and the reference scanning line data having the scanning angle adjacent to the scanning angle of the first scanning line data including the guidewire artifact information to obtain the dynamic coefficient.

For the specific limitations of the guidewire artifact suppression apparatus, reference can be made to the limitations of the guidewire artifact suppression method mentioned above, which will not be repeated here. Modules in the above-mentioned guidewire artifact suppression apparatus can be fully or partially implemented by software, hardware or a combination thereof. The above-mentioned modules may be embedded in or independent of a processor in a computer device in the form of hardware, or may be stored in a memory in a computer device in the form of software, so that the processor can invoke and execute operations corresponding to the modules.

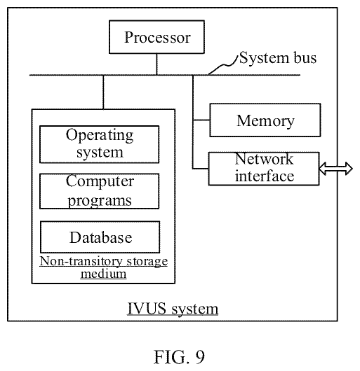

In an embodiment, an IVUS system is provided, and an internal structure diagram thereof may be as shown in FIG. 9. The computer device may include a processor, a memory, a network interface and a database connected to each other via a system bus. The processor of the computer device is configured to provide computing and control capabilities. The memory of the computer device includes a non-transitory storage medium and an internal memory. The non-transitory storage medium stores an operating system, a computer program, and a database. The internal memory provides an environment for the operations of the operating system and computer programs in the non-transitory storage medium. The database of the computer device is configured to store the first scan line data. The network interface of the computer device is configured to communicate with an external terminal via a network connection. The computer program, when executed by a processor, causes the processor to implement a guidewire artifact suppression method.

Those skilled in the art may understand that the structure shown in FIG. 9 is merely a block diagram of a partial structure related to the solution of the present disclosure, and does not constitute a limitation on the computer device to which the solution of the present disclosure is applied. The specific computer device may include more or fewer components than shown in the figure, or combine certain components, or have a different arrangement of components.

In an embodiment, a computer-readable storage medium is provided, on which a computer program is stored, and the computer program, when executed by a processor, causes the processor to implement the following steps of:
acquiring first scan line data;
when the first scan line data includes guidewire artifact information, performing filtering processing and tissue information extraction processing on the first scan line data to obtain second scan line data;
performing logarithmetics processing on the second scan line data based on a dynamic coefficient to obtain third scan line data for reconstructing an ultrasound image, in which the dynamic coefficient is obtained based on the second scan line data, a position of the guidewire artifact information and reference scan line data, and the reference scan line data is obtained based on the first scan line data excluding the guidewire artifact information,
In an embodiment, the computer program, when executed by a processor, may causes the processor to further implement the following steps of:
performing the filtering processing and the tissue information extraction processing on the first scan line data when the first scan line data excludes the guidewire artifact information, to obtain fourth scan line data;
performing the logarithmetics processing on the fourth scan line data based on a fixed coefficient to obtain fifth scan line data for reconstructing the ultrasound image.

In an embodiment, when the step of performing the filtering processing on each piece of first scan line data is executed by the processor, the processor may further implement the following steps of:
performing bandpass filtering processing on the first scan line data when the first scan line data excludes the guidewire artifact information;
performing the bandpass filtering processing and bandstop filtering processing on the first scan line data respectively when the first scan line data includes the guidewire artifact information.

In an embodiment, the computer program, when executed by a processor, may cause the processor to further implement the following steps of:
processing a time domain, a frequency domain or a time-frequency domain of each piece of first scan line data to obtain a characteristic value of each piece of first scan line data;
processing each characteristic value by a classifier to obtain a classification result;
determining whether each piece of first scan line data includes the guidewire artifact information according to the classification result.

In an embodiment, when the step of processing each characteristic value by the classifier is executed by the processor, the processor may further implement the following steps of:
processing characteristic values of the first N pieces of sub-data by the classifier, in which a value of N is obtained according to setting parameters and a catheter structure of a ultrasound system.

In an embodiment, when the step of processing each characteristic value by the classifier is executed by the processor, the processor may further implement the following steps of
processing the characteristic values of the plurality of pieces of sub-data in sequence by the classifier until a guidewire artifact determination event occurs, in which the guidewire artifact determination event may include that the first scan line data is determined to include the guidewire artifact information based on a classification result corresponding to any piece of sub-data.

In an embodiment, the computer program, when executed by a processor, may cause the processor to further implement the following steps of
determining the reference scan line data as a standard template;
determining theoretical data of a scanning point according to the position of the guidewire artifact information, the standard template, and actual data of the scanning point in the first scanning line data including the guidewire artifact information, and
determining the dynamic coefficient based on the theoretical data and the actual data.

In an embodiment, the computer program, when executed by a processor, may cause the processor to further implement the following steps of
acquiring a fitting function by a neural network algorithm model;
processing, by the fitting function, the position of the guidewire artifact information, the actual data of the scanning point in the first scanning line data including the guidewire artifact information, and the reference scanning line data having a scanning angle adjacent to the scanning angle of the first scanning line data including the guidewire artifact information to obtain the dynamic coefficient.

A person of ordinary skill in the art can understand that all or part of the processes in the above-mentioned embodiment methods can be implemented by instructing related hardware through a computer program. The computer program can be stored in a non-transitory computer-readable storage medium. When the computer program is executed, it can include the processes of the embodiments of the above-mentioned method. Any reference to memory, storage, database or other media used in the embodiments provided in the present disclosure may include a non-transitory memory and/or transitory memory. The non-transitory memory may include a read-only memory (ROM), a programmable ROM (PROM), an electrically programmable ROM (EPROM), an electrically erasable programmable ROM (EEPROM), or a flash memory. The transitory memory may include a random access memory (RAM) or an external cache memory. By way of illustration and not limitation, RAM is available in many forms, such as static RAM (SRAM), dynamic RAM (DRAM), synchronous DRAM (SDRAM), double data rate SDRAM (DDRSDRAM), enhanced SDRAM (ESDRAM), synchlink DRAM (SLDRAM), rambus DRAM (RDRAM), and direct rambus DRAM (DRDRAM), etc.

The technical limitations in the above-described embodiments may be arbitrarily combined. To make the description concise, all possible combinations of the technical limitations in the above-described embodiments are not described. However, as long as there is no contradiction in combinations of these technical limitations, these combinations should be considered to be within the scope of the present disclosure.

The above-described embodiments merely express several implementation modes of the present disclosure, and the description thereof is relatively specific and detailed, but it should not be construed as limiting the scope of the present disclosure. It should be pointed out that, those skilled in the art can make several modifications and improvements without departing from the concept of the present disclosure, which all fall within the scope of protection of the present disclosure. Therefore, the protection scope of the present disclosure shall be subject to the appended claims.

1. A guidewire artifact suppression method, comprising:
acquiring first scan line data;
when the first scan line data includes guidewire artifact information, performing filtering processing and tissue information extraction processing on the first scan line data to obtain second scan line data; and
performing logarithmetics processing on the second scan line data based on a dynamic coefficient to obtain third scan line data for reconstructing an ultrasound image, wherein the dynamic coefficient is obtained based on the second scan line data, a position of the guidewire artifact information and reference scan line data, and the reference scan line data is obtained based on first scan line data excluding the guidewire artifact information.

2. The guidewire artifact suppression method according to claim 1, further comprising:
performing the filtering processing and the tissue information extraction processing on the first scan line data when the first scan line data excludes the guidewire artifact information, to obtain fourth scan line data; and
performing the logarithmetics processing on the fourth scan line data based on a fixed coefficient to obtain fifth scan line data for reconstructing the ultrasound image.

3. The guidewire artifact suppression method according to claim 2, wherein the performing the filtering processing on the first scan line data comprises:
performing bandpass filtering processing on the first scan line data when the first scan line data excludes the guidewire artifact information; and
performing the bandpass filtering processing and bandstop filtering processing on the first scan line data respectively when the first scan line data includes the guidewire artifact information.

4. The guidewire artifact suppression method according to claim 1, further comprising:
processing a time domain, a frequency domain or a time-frequency domain of each piece of first scan line data to obtain a characteristic value of each piece of first scan line data;
processing each characteristic value by a classifier to obtain a classification result; and
determining whether each piece of first scan line data includes the guidewire artifact information according to the classification result.

5. The guidewire artifact suppression method according to claim 4, wherein the first scan line data comprises a plurality of pieces of sub-data; and
the processing each characteristic value by the classifier comprises:
processing characteristic values of first N pieces of sub-data by the classifier, wherein a value of N is obtained according to setting parameters and a catheter structure of an ultrasound system.

6. The guidewire artifact suppression method according to claim 4, wherein the first scan line data comprises a plurality of pieces of sub-data; and
the processing each characteristic value by the classifier comprises:
processing characteristic values of the plurality of pieces of sub-data in sequence by the classifier until a guidewire artifact determination event occurs, wherein the guidewire artifact determination event comprises determining that the first scan line data includes the guidewire artifact information based on a classification result corresponding to a piece of sub-data.

7. The guidewire artifact suppression method according to claim 1, further comprising:
determining the reference scan line data as a standard template;
determining theoretical data of a scanning point according to the position of the guidewire artifact information, the standard template, and actual data of the scanning point in the first scanning line data including the guidewire artifact information; and
determining the dynamic coefficient based on the theoretical data and the actual data.

8. The guidewire artifact suppression method according to claim 1, further comprising:
acquiring a fitting function by a neural network algorithm model;
processing, by the fitting function, the position of the guidewire artifact information, the actual data of the scanning point in the first scanning line data including the guidewire artifact information, and the reference scanning line data having a scanning angle adjacent to a scanning angle of the first scanning line data including the guidewire artifact information to obtain the dynamic coefficient.

9. The guidewire artifact suppression method according to claim 1, wherein the performing the logarithmetics processing on the second scan line data based on the dynamic coefficient to obtain the third scan line data for reconstructing the ultrasound image comprises obtaining the third scan line data based on the following formula:
$$y = log ( k * x + 1 ) ,$$
where y denotes the third scan line data, k denotes the dynamic coefficient, and x denotes the second scan line data.

10. A guidewire artifact suppression apparatus, comprising:
a data cache module configured to acquire first scan line data;
a processing module configured to, when the first scan line data includes guidewire artifact information, perform filtering processing and tissue information extraction processing on the first scan line data to obtain second scan line data; and
a logarithmetics module configured to perform logarithmetics processing on the second scan line data based on a dynamic coefficient to obtain third scan line data for reconstructing an ultrasound image, wherein the dynamic coefficient is obtained based on the second scan line data, a position of the guidewire artifact information and reference scan line data, and the reference scan line data is obtained based on the first scan line data excluding the guidewire artifact information.

11. An intravascular ultrasound (IVUS) system, comprising a processor and a memory storing a computer program, wherein the processor, when executing the computer program, implements the method of claim 1.

12. A computer-readable storage medium, on which a computer program is stored, wherein the computer program, when executed by a processor, causes the processor to implement the method of claim 1.
